(19) United States
(12) Patent Application Publication
(10) Pub. No.: US 2021/0350915 A1
(43) Pub. Date: Nov. 11, 2021

(54) UNIVERSAL PHYSICIAN RANKING SYSTEM BASED ON AN INTEGRATIVE MODEL OF PHYSICIAN EXPERTISE

(71) Applicant: DocOne Inc., New York, NY (US)

(72) Inventor: Kresimir Letinic, New York, NY (US)

(73) Assignee: DocOne Inc., New York, NY (US)

(21) Appl. No.: 17/384,879

(22) Filed: Jul. 26, 2021

Related U.S. Application Data
(60) Provisional application No. 63/056,130, filed on Jul. 24, 2020.

(51) Int. Cl. G16H 40/20; G06N 3/04; G16H 70/20; G16H 70/60; G16H 50/70; G16H 50/20; G06F 40/30

(52) U.S. Cl. CPC G16H 40/20; G06N 3/0427; G16H 70/20; G06F 40/30; G16H 50/70; G16H 50/20; G16H 70/60

(57) ABSTRACT

Systems and methods for measuring physician expertise are disclosed. Furthermore, systems and methods for ranking physicians based on said measure of expertise are disclosed. The systems and methods may comprise (a) capturing and mining large-scale, up-to-date medical and clinical knowledge sources (e.g., biomedical corpora, clinical guidelines, clinical trials, professional physician data); (b) building models of medical conditions that link each condition to relevant concepts (e.g., specialties, medical procedures, drug regimens) and relevant clinical research (e.g., published articles, clinical trials); (c) transforming biomedical concepts (e.g., conditions, procedures, drugs) extracted from text in natural language into terms and codes of biomedical ontologies; (d) enriching mission-critical biomedical ontologies and creating mappings between them; (e) matching physician data against a medical condition model in order to rank physicians according to their relevance to a given condition.

### CROSS-REFERENCE TO RELATED APPLICATIONS

This application claims priority to U.S. Provisional Patent Application No. 63/056,130, filed Jul. 24, 2020 and entitled, “EXTRACTING AND APPLYING MEDICAL KNOWLEDGE TO MODELS OF PHYSICIAN EXPERTISE”, which is incorporated herein by reference in its entirety.

### BACKGROUND

Advancements in technology have had an undeniable impact on the practice of medicine. For example, the equipment that resides in doctor's offices and operating rooms has become significantly more advanced in recent years. From complex robotic-based surgery to enhanced image capture and processing tools, few other areas have received the same benefit from technology as the medical field.

However, the increased use and development of technology has not enhanced all aspects of medical care. A particular area that is lagging behind is that of patient resources. Currently, most people who believe they may be suffering from a medical condition initiate the process of diagnosis with an internet search. Often, the internet search leads them to a website that tries to match their symptoms to one or more potential conditions. However, almost all of these “personal diagnosis” tools are imprecise and misleading.

Moreover, no system exists for consumers and patients to intelligently select a doctor. Current solutions involve going to a primary care physician and requesting a recommendation, asking trusted friends/colleagues for advice, or simply trying to search for specialists in certain medical fields with little guidance. In some cases, an individual with insurance coverage may be able to search for a specialist via their insurance company's website, or an individual who is a member of a health system may be able to search via their system's website. In other cases, individuals may search for a specialist using third-party physician directories. However, directories that exist today provide only basic information about physicians, such as their specialty and location. No meaningful measure of physician expertise or clinical focus is provided. Some directories rely on patient satisfaction reviews, but such reviews were shown to have no correlation with physician expertise. The National Assessment of Adult Literacy found that 88% of U.S. adults lack proficient health literacy, or the ability to navigate healthcare. The most highly cited need in healthcare consumers studies is assistance with finding the right physician.

Furthermore, medical knowledge has been expanding rapidly, with a study in Transactions of the American Clinical and Climatological Association estimating that medical knowledge doubles every 73 days. The result is niche specialization in physician expertise which is far beyond the ability of an average primary care physician, let alone a patient, to track. As a result, it is difficult for referring physicians to find the right specialists for their patients. Physicians regard clinical expertise as the most important factor in their referral decisions, but they lack reliable, transparent and precise metric for specialist expertise. Furthermore, most physicians are not certain if their referrals are correct. According to the Kyruus Physician Referral Survey, more than 75% of specialists received wrong referrals within the past year, with at least 25% of all referrals being clinically inappropriate. Physician referrals often direct patients to physicians that are familiar to referring physicians, or “in the family”, rather than to those with the right expertise.

Thus, a solution is needed that enables a user to more accurately search for and find the physician with the best expertise to diagnose and treat the patient. Specifically, the solution should comprise systems and methods for mining large, ever-expanding corpora of biomedical literature and comprehensive other sources of biomedical and clinical data, building accurate models of medical conditions (which includes linking medical conditions to relevant symptoms, medical, surgical, radiology and pathology procedures, drug regimens, research studies, publications, clinical trials, quality measures etc.), mapping comprehensive physician data to the models of medical conditions, and scoring, in a clinically relevant manner, the expertise of physicians in regard to specific medical conditions.

### SUMMARY

This summary is provided to comply with 37 C.F.R. § 1.73, requiring a summary of the invention briefly indicating the nature and substance of the invention. It is submitted with the understanding that it will not be used to interpret or limit the scope or meaning of the present disclosure.

A system for producing a clinically relevant measure of physician expertise is provided. The system includes a processor and a non-transitory, computer-readable storage medium in operable communication with the processor. The computer-readable storage medium contains one or more programming instructions that, when executed, cause the processor to receive physician's data relating to one or more physicians; receive one or more datasets of biomedical corpora, clinical guidelines, clinical trials databases, biomedical ontologies, and other clinical resources; receive input comprising at least one of one or more medical conditions and one or more symptoms; determine one or more subsets of the one or more datasets relating to at least one of the one or more physicians; generate one or more semantically similar terms to the input; generate innovation information comprising a statistical analysis of the input and an appearance of the one or more semantically similar terms in the one or more subsets; generate experience information comprising a statistical analysis of the context relating to the input within the one or more subsets; generate innovation scores, for each of the one or more physicians, based on the innovation information; generate experience scores, for each of the one or more physicians, based on the experience information; and score the one or more physicians' expertise with respect to the medical condition based on a combination of the innovation scores and the experience scores.

According to certain embodiments, the one or more programming instructions that, when executed, cause the processor to score the one or more physicians' expertise with respect to the medical condition based on a combination of the innovation scores and the experience scores include one or more programming instructions that, when executed, cause the processor to score the one or more physicians' expertise further based on an authority score.

According to certain embodiments, the one or more programming instructions that, when executed, cause the processor to score the one or more physicians' expertise with respect to the medical condition based on a combination of the innovation scores and the experience scores include one or more programming instructions that, when executed, cause the processor to score the one or more physicians' expertise further based on a quality score.

According to certain embodiments, the one or more programming instructions that, when executed, cause the processor to score the one or more physicians' expertise with respect to the medical condition based on a combination of the innovation scores and the experience scores include one or more programming instructions that, when executed, cause the processor to score the one or more physicians' expertise further based on a team score.

According to certain embodiments, the one or more programming instructions that, when executed, cause the processor to determine one or more subsets of the one or more datasets relating to at least one of the one or more physicians further comprise one or more programming instructions that, when executed, cause the processor to perform name disambiguation on each of the one or more physicians.

According to certain embodiments, the one or more programming instructions that, when executed, cause the processor to generate an innovation score further comprise one or more programming instructions that, when executed, cause the processor to calculate a term frequency for at least one of the inputs and the one or more semantically similar terms.

According to certain embodiments, the one or more programming instructions that, when executed, cause the processor to generate one or more semantically similar terms to the input further comprise one or more programming instructions that, when executed, cause the processor to exploit the taxonomical structure of the Medical Subject Headings ontology.

According to certain embodiments, the statistical analysis of the context relating to the input is performed using at least one of the following algorithms: shallow neural models, deep learning models, natural language processing, word2vec, GloVE, biowordvec, cui2vec, transformer-based models, BERT, BioBERT, T5, and BigBird.

According to certain embodiments, the statistical analysis of the context relating to the input comprises determining guidelines, procedures, and drug regimens for diagnosing or treating the input.

According to certain embodiments, the statistical analysis of the context relating to the input comprises mapping UMLS procedure concepts onto CPT codes.

A method for producing a clinically relevant measure of physician expertise is provided. The method includes receiving physician's data relating to one or more physicians, receiving one or more datasets of biomedical corpora, clinical guidelines, clinical trials databases, biomedical ontologies, and other clinical resources, receiving input comprising at least one of one or more medical conditions and one or more symptoms, determining one or more subsets of the one or more datasets relating to at least one of the one or more physicians, generating one or more semantically similar terms to the input, generating innovation information comprising a statistical analysis of the input and an appearance of the one or more semantically similar terms in the one or more subsets, generating experience information comprising a statistical analysis of the context relating to the input within the one or more subsets, generating innovation scores, for each of the one or more physicians, based on the innovation information, generating experience scores, for each of the one or more physicians, based on the experience information, and scoring the one or more physicians' expertise as it relates to the medical condition based on a combination of the innovation scores and the experience scores.

According to certain embodiments, scoring the one or more physicians' expertise with respect to the medical condition based on a combination of the innovation scores and the experience scores further comprises scoring the one or more physicians' expertise further based on an authority score.

According to certain embodiments, scoring the one or more physicians' expertise with respect to the medical condition based on a combination of the innovation scores and the experience scores further comprises scoring the one or more physicians' expertise further based on a quality score.

According to certain embodiments, scoring the one or more physicians' expertise with respect to the medical condition based on a combination of the innovation scores and the experience scores further comprises scoring the one or more physicians' expertise further based on a team score.

According to certain embodiments, determining one or more subsets of the one or more datasets relating to at least one of the one or more physicians further comprises performing name disambiguation on the one or more physicians.

According to certain embodiments, generating an innovation score comprises calculating a term frequency for at least one of the inputs and the one or more semantically similar terms.

According to certain embodiments, generating one or more semantically similar terms to the input comprises exploiting the taxonomical structure of the Medical Subject Headings ontology.

According to certain embodiments, the statistical analysis of the context relating to the input comprises at least one of the following algorithms: shallow neural models, deep learning models, natural language processing, word2vec, GloVE, biowordvec, cui2vec, transformer-based models, BERT, BioBERT, T5, and BigBird.

According to certain embodiments, the statistical analysis of the context relating to the input comprises determining guidelines, procedures, and drug regimens for diagnosing or treating the input.

According to certain embodiments, the statistical analysis of the context relating to the input comprises mapping UMLS procedure concepts onto CPT codes.

### BRIEF DESCRIPTION OF THE DRAWINGS

For the purpose of illustrating the invention, there is shown in the drawings various embodiments; it being understood, however, that the invention is not limited to the specific instrumentalities disclosed as they are used for illustrative purposes only. Included in the drawings are the following Figures:

FIG. 1 depicts a diagram of an illustrative example of a scoring methodology for a physician for a given medical condition in accordance with an embodiment

FIG. 2 depicts a diagram of essential elements enabling linking conditions and physicians in the integrative model, in accordance with an embodiment

FIG. 3A depicts an illustrative example of the Medical Subject Headings hierarchy.

FIG. 3B depicts another illustrative example of the Medical Subject Headings hierarchy.

FIG. 4 depicts an illustrative mapping between multiple biomedical ontologies, in accordance with an embodiment.

FIG. 5 depicts an illustrative diagram of a method for determining a physician experience score, in accordance with an embodiment.

FIG. 6 depicts an illustrative diagram of hubs and authorities of a referral network, in accordance with an embodiment.

FIG. 7 depicts an illustrative block diagram of a method for determining a physician team score, in accordance with an embodiment.

FIG. 8 depicts an illustrative data processing system in accordance with an embodiment.

### DETAILED DESCRIPTION

The present description and claims may make use of the terms “a,” “at least one of,” and “one or more of,” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that more than one of such features/elements be present. To the contrary, these terms/phrases only require at least a single feature/element with the possibility of multiple of such features/elements being within the scope of the description and claims.

In addition, it should be appreciated that the following description uses multiple examples for various elements of the illustrative embodiments to further illustrate example implementations of the illustrative embodiments and to aid in the understanding of the mechanisms of the illustrative embodiments. These examples are intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the example provided herein without departing from the spirit and scope of the present invention.

The systems and methods described herein rely on input from a large spectrum of data sources encompassing the entire biomedical corpora and other medical and clinical resources. Illustrative data sources include PubMed, PubMed Central (PMC), the National Institute of Health (NIH), the National Cancer Institute (NCI), HemOnc.org, The U.S. National Library of Medicine Clinical Trial Database, Centers for Medicare & Medicaid Services (CMS), the Agency for Healthcare Research and Quality (AHRQ), and HealthData.gov. Anonymized medical records and claims data from healthcare providers (e.g., physicians and hospitals) and insurers may also be used when available. The Unified Medical Language Systems (UMLS) ontology greatly assists in integrating the many biomedical taxonomies used by these sources by providing a unified global vocabulary and a mapping structure between that vocabulary and each integrated biomedical taxonomy (e.g., Current Procedural Terminology (CPT) codes, ICD-10-CM, LOINC, Medical Subject Headings (MeSH), RxNorm, SNOMED-CT). UMLS includes the Metathesaurus which stores all the concepts and links similar names for the same concept from nearly 200 different vocabularies, and the Semantic Network which consists of semantic types that categorize all concepts represented in the Metathesaurus (e.g., ‘Disease or Syndrome,’ ‘Sign or Symptom’) and semantic relations between semantic types. The Metathesaurus contains a concept unique identifier (CUI) for each concept. Each CUI may have associated lexical variants of the concept, which are each given a lexical unique identifier (LUI). Each LUI may have associated variations in language and punctuation that are each given a string unique identifier (SUI) and entries for how the LUI is identified in a taxonomy in an atom unique identifiers (AUI).

The systems and methods provide an integrative model of physician expertise relative to thousands of medical conditions, which forms the basis of a physician ranking and recommendation platform. As taught herein, natural language processing (NLP) methods are used for mining large scale corpuses of biomedical literature and other types of medical/clinical information expressed in unstructured natural language. Furthermore, methods for transforming unstructured information extracted in natural language into structured biomedical ontologies are mission-critical. A person of ordinary skill in the art will understand that many NLPs may be applied alternatively or in conjunction with the NLPs disclosed herein with varying levels of success.

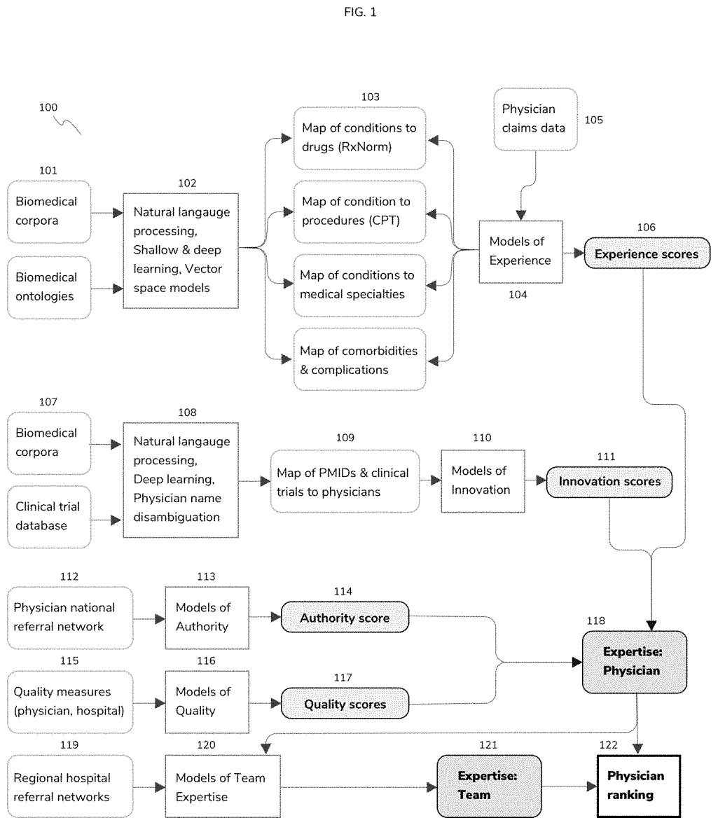

FIG. 1 depicts an illustrative block diagram for ranking a physician in regard to a medical condition 100 in accordance with an embodiment. In some embodiments, the physician's individual expertise score 118 may comprise a combination of other scores including an experience score 106, an innovation score 111, an authority score 114, and a quality score 117.

In some embodiments, the experience score 106 may be determined using one or more models of experience 104. The models 104 may receive input comprising physicians claim data 105 and one or more mappings 103 from conditions to drug regimens, procedures, medical specialties, comorbidities, and complications. The mappings 103 may be derived using natural language processing, deep learning, shallow learning, and vector space models 102 applied to biomedical corpora and one or more biomedical ontologies 101.

In some embodiments, the innovation score 111 may be determined using one or more models of innovation 110. The models 110 may be based on mappings 109 from physicians to clinical trials and research publications (PMIDs). The mappings 109 may be derived from biomedical corpora and clinical trial databases 107 using natural language processing, deep learning, and physician name disambiguation 108.

In some embodiments, the authority score 114 may be determined using one or more models of authority 113. The models 113 may be based on data from the physicians referral network 112.

In some embodiments, the quality score 117 may be determined using one or more models of quality 116. The models may be based on quality measures 115 from the physician or hospital.

In some embodiments, the physician may also comprise a team expertise score 121, which may comprise the individual expertise scores 118 of other physicians within some network or proximity of the target physician (e.g., a Hospital Referral Region (HRR) 119). In some embodiments, the team expertise score 121 may be at least partially derived from a referral graph of the physician in question 120.

In some embodiments, combination of a physician's individual expertise score 118 and the team expertise score 121 may form a final rank 122 for the physician. In further embodiments, any of the scores combined to form the final rank 122, the physician's individual expertise score 118, and/or the team expertise score 121 may be individually weighted.

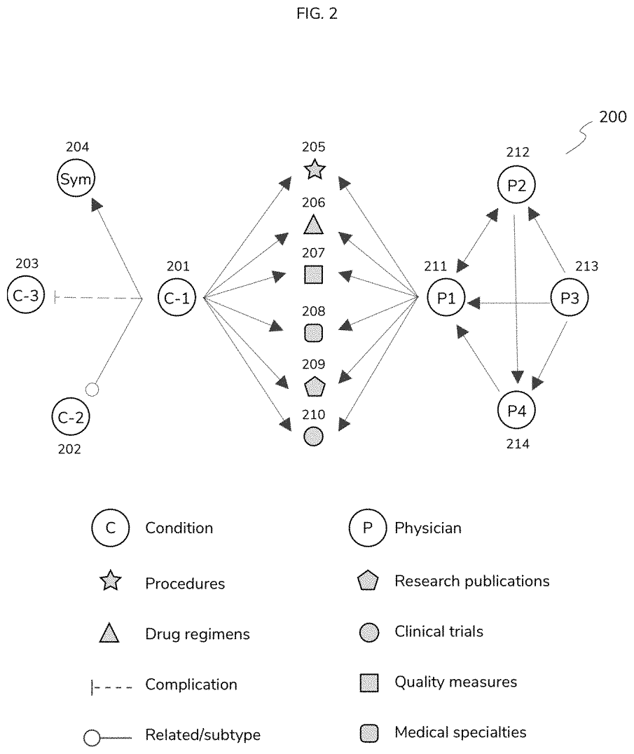

FIG. 2 illustrates the relationships between a physician and a medical condition 200 in accordance with an embodiment. Specifically, FIG. 2 illustrates data types linking a physician P1 211 with a medical condition C-1 201.

In many embodiments, physician P1 211 may exist in a referral network comprising multiple physicians. For example, in FIG. 2, physician P1 211 has received referrals from physicians P2 212, P3 213, and P4 214, and referred patients to physician P2 212.

In some embodiments, medical condition C-1 201 may have one or more symptoms (e.g., Sym 204). In some embodiments, medical condition C-1 has a comorbidity and/or complication associated with one or more other medical conditions (e.g., C-3 203). In many embodiments, C-1 is closely related to one or more other medical conditions. C-1 may be a subtype of another medical condition (e.g., C-2 202). C-1 may also or additionally be a parent type of one or more other medical conditions.

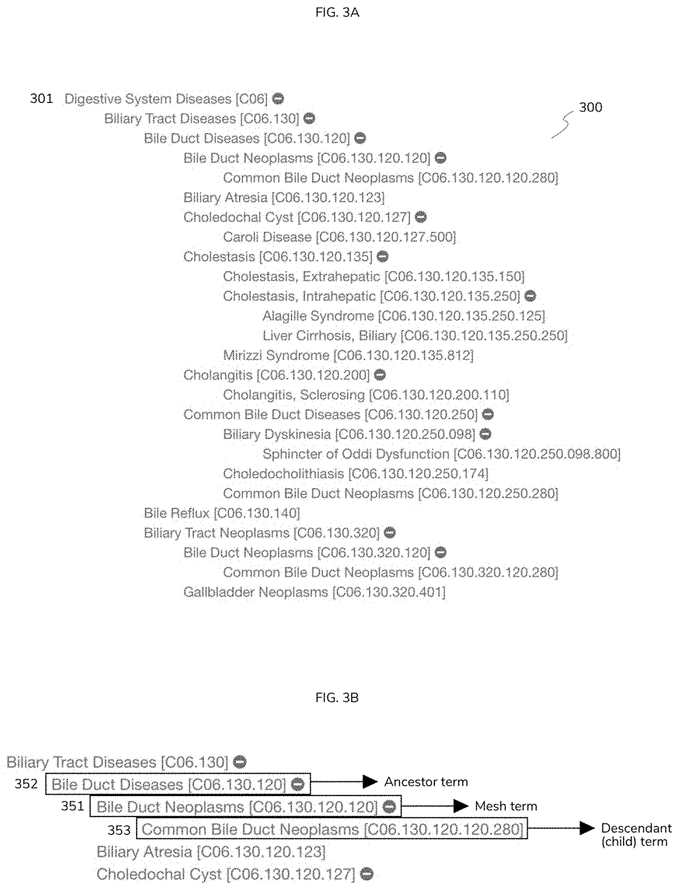

Briefly referring to FIG. 3A, illustrative hierarchies of medical conditions are depicted according to the MeSH ontology 300. Specifically, hierarchies for Digestive System Diseases 301 are depicted. MeSH acts a biomedical thesaurus for indexing research articles and clinical trials. FIG. 3B depicts a portion of the ontology for Biliary Tract Disease 350. A given MeSH term 351 has a hierarchy coding such as C06.130.120.120 (Bile Duct Neoplasms) which encodes its relationship to other terms. A term 351 may have a parent 352 condition and/or one or more child conditions 353 which are subtypes of the term. Each period in the code indicates an ancestral generation from the category of the ontology.

Referring back to FIG. 2, relationships may be determined between physician P1 211 and medical condition C-1 201 based on multiple factors and sources. These factors and sources may include diagnostic and treatment procedures 205, drug regimens 206, quality measures 207, medical specialties or subspecialties 208, research publications 209, and clinical trials 210.

### Innovation Modeling

Physician innovation modeling captures and scores the physician's contribution to the field of medicine, specifically in regard to the medical condition under consideration. Information-theoretic approaches (e.g., semantic similarity, term frequency, and inverse document frequency) as well as exploiting the design of some medical ontologies are explored herein. In some embodiments, these approaches may be applied to any source of biomedical corpora and/or clinical trial data to identify and score relevant terminology in the source. In some embodiment different classifications of source documents (e.g., publication vs. clinical trial) may be scored separately towards producing several different innovation scores for the physician.

As disclosed above, in reference to FIG. 3B, the MeSH ontology encodes medical conditions as parents, siblings, and/or children of other medical conditions. A child is a subtype of the parent. In the MeSH ontology, a parent's code may be determined by truncating the digits after the last decimal point in a child's code. In some embodiments, the innovation score for a physician is based on a weighted sum of counts of query-related MeSH terms. In some embodiments, the weighted sum includes not only the query, but other query-related terms, expanding the scope by keeping all relevant terms. In some embodiments, query-related terms are terms located on the branch of the MeSH tree that contains the query. In some embodiments, semantic similarity measure between terms is used to determine query-related MeSH terms. In some embodiments, the query and query-related terms may each be individually weighted in the weighted sum.

In further embodiments, alternate and/or additional ontologies may be used to further expand the scope of query-related terms. For example, the one or more query-related terms used in MeSH may be converted to UMLS and associated LUIs, SUIs and AUIs may also be searched.

In some embodiments, semantic similarity values are calculated between the query term and query-related terms and are used as the weights in a weighted sum. In some embodiments, the semantic similarity measure comprises path- and/or depth-based measures which, for example, consider the position of a query-related term with respect to the position of the query term in the taxonomy. In such embodiments, the semantic similarity measure may be determined based on the position of each of the query term and the query-related term relative to the position of their most specific common ancestor or least common subsumer (LCS).

In some embodiments, the semantic similarity measure comprises information content (IC)-based measures in which a value for the IC of the LCS is divided by the sum of values for the ICs of the individual terms.

In some embodiments, the semantic similarity measure comprises computing an IC value for a term, where the IC value of a term depends on the depth of terms that the term subsumes and the relative depth of the term itself. In some embodiments, an ancestor's subgraph-based IC quantification (AIC) may be used. In such embodiments, the IC value of a term may be modeled by the subgraph formed by its ancestors. A term depends strongly on its direct hypernyms/parents and ancestors, as the term inherits the basic features from the ancestor term and adds its distinctive features to form its own semantics. The contribution, or score of an ancestor is based on the specificity of the ancestor's direct hypernyms. Each hypernym's contribution to the “score” of an ancestor increases with the hypernym's depth in the ontology and its specificity, which in turn depends on the depth of its descendants.

In some embodiments, physician innovation scores are calculated using key words (e.g., MeSH terms) indexed and/or extracted from articles published by the physician in question, or clinical trials led by the physician in question. For each relevant article or clinical trial of the physician, a value is assigned to each relevant MeSH term according to predetermined value-assigning rules. In further embodiments, the value-assigning rules may comprise identifying the type of published article (e.g., a guideline) or clinical trial (e.g., phase), and the ordering of authors in the author list (e.g., first, or last author).

### Experience Modeling

Physician experience modeling captures and scores the physician's experience in diagnosing and treating a given medical condition. Some embodiments may comprise natural language processing, shallow and/or deep learning language models, and vector space models of information retrieval.

In many embodiments, the system must evaluate an assortment of biomedical corpora for context in regard to procedures, drug regimens, medical specialties, comorbid conditions and complications relevant to medical conditions. In some embodiments, the system evaluates official medical guidelines associated with the medical condition.

In some embodiments, one or more shallow learning models and/or deep learning approaches may be employed for linking conditions to relevant procedures and drug regimens. In some embodiments, the system uses a shallow language learning model pretrained on biomedical corpora. In further embodiments, the model is used to generate embeddings, or vector representations for individual concepts, as well as sensible concatenated combinations of concepts in the CPT code taxonomy and MeSH disease ontology. In some embodiments, a cosine similarity between CPT and MeSH embeddings aid in selecting the most relevant procedures for any given condition and create a MeSH-to-CPT map.

In another embodiment, the system utilizes deep learning language models based on bidirectional transformers pre-trained using large scale unannotated general domain and biomedical corpora. In some embodiments, the models are subsequently fine-tuned on general domain and biomedical datasets, including proprietary datasets, for supervised NLP tasks. In some embodiments, the system uses these models to extract, from biomedical literature (e.g., PubMed, PMC, guidelines, etc.), all relevant procedures and drug regimens for any given medical condition. In further embodiments, the drug regiments extracted in natural language may be transformed into RxNorm concepts. Similarly, medical procedures extracted in natural language may be transformed into UMLS procedure concepts.

In some embodiments, the word2vec algorithm may be implemented to achieve this goal. The word2vec algorithm utilizes vector space models to represent or embed words in a continuous vector space. Semantically similar words are embedded near each other. Specifically, the word2vec algorithm is a feed-forward shallow neural net trained to reconstruct linguistic contexts of words and produce a vector space. Given a target word, the Skip-gram neural network model, central to the word2vec algorithm, predicts the surrounding context.

In some embodiments, the Global Vectors for Word Representation (GloVe) algorithm, an unsupervised learning algorithm for obtaining word vectors that capture meaning, may be used alternatively or in addition to other language models. The GloVe algorithm uses global count statistics and local information to compute a co-occurrence matrix using a fixed window. The difference in word vectors predicts co-occurrence ratios. Specifically, GloVe fits a weighted log-linear model to co-occurrence statistics. Given that a target word w and a context word c co-occur y times, GloVe solves a least-squares optimization problem to generate word embeddings.

In some embodiments, the BioWordVec algorithm, a biomedical word embeddings model that combines subword information from unlabeled biomedical text with MeSH vocabulary, may be used alternatively or addition to other language models. The previously described algorithms, word2vec and GloVe, ignore the internal structure of words and therefore are not good at learning rare, out of vocabulary, or evolving words. The subword embedding model, in BioWordVec, makes use of the representations of character n-grams to address this limitation. Subword embedding model can share the character n-gram representations between PubMed/PMC text words and MeSH terms, thereby integrating PubMed/PMC and MeSH into a unified embedding space.

In some embodiments, the cui2vec algorithm trained on large and multi-modal sources of medical data, and its comprehensive set of resulting clinical embeddings, may be used alternatively or in addition to other language models. The cui2vec algorithm creates co-occurrence matrices and factors them using GloVe, or derives shifted positive pointwise mutual information matrices and factors them using singular value decomposition to create word2vec-style embeddings.

Context-free models (word2vec, GloVe) learn a single representation for every word. Deeply contextual models learn representations that change based on the word's context.

In some embodiments, the system uses the contextual model Bi-directional Encoder Representations from Transformers (BERT), a deep bidirectional unsupervised language model trained using unannotated text from BookCorpus and Wiki corpus. While standard language models are unidirectional, such models are sub-optimal for NLP tasks that require context from both directions. BERT is trained on a masked language modeling (MLM) task that allows the representation to fuse the left context and the right context. BERT features multi-head attention which enables the model to capture a broad range of relationships between words. The encoder in BERT is a stack of N identical layers in which each layer has two sublayers: multi-head self-attention and a position-wise fully connected feed-forward network.

The BERT pre-trained model weights encode significant amounts of information about language. As a result, BERT can be further fine-tuned with just one additional layer. All BERT parameters and added output layer are fine-tuned jointly (few new parameters are learned). In some embodiments, BERT can be fine-tuned to answer questions written in natural language given related passages. In some embodiments, The Stanford Question Answering Dataset (SQuAD), which comprises around one hundred thousand crowd-sourced question/answer pairs based on Wikipedia articles, may be used to fine-tune BERT for a question answering task.

In further embodiments, the system uses BioBERT, a domain-specific BERT model pre-trained on both general, Wiki and Books corpora and biomedical, PubMed/PMC corpora (PubMed abstracts, PMC full text articles; 4.5B words in PubMed and 13.5B words in PMC).

General language models trained only on general corpora perform poorly on biomedical text mining tasks. Furthermore, deep learning generally requires large amounts of training data. In the biomedical domain, the construction of a large training set is very costly as it requires the use of specialized, hard-to-find experts in various biomedical fields. Because of this, only a small amount of labeled training data is available for biomedical text mining. BioBERT leverages transfer learning to address both issues. It is pre-trained on a large quantity of unlabeled biomedical text. It requires only a minimal number of task-specific parameters for fine-tuning. BioBERT provides an accurate biomedical text mining tool, outperforming BERT and current state-of-the-art models in biomedical NLP.

In some embodiments, BioBERT may be further refined as Clinical BioBERT, which is specifically trained to analyze clinical narratives (e.g., physician notes). Clinical narratives have known differences in linguistic characteristics from both general text and non-clinical biomedical text, motivating the need for specialized clinical BERT models. Clinical BioBERT is trained on clinical text from the approximately two million notes in the MIMIC-III v1.4 clinical database. Clinical BioBERT comprises two models which have been trained using this database: Clinical BERT, which uses text from all note types, and Discharge Summary BERT, which uses only discharge summaries. On some clinical NLP tasks, Clinical BioBERT shows improvements over BioBERT or BERT.

In some embodiments, other transformer-based models may be used for analyzing context in language. In some embodiments, a model with encoder-decoder architecture may be used. In other embodiments, the T5 encoder-decoder transformer may be used. T5 treats every NLP problem as a text-to-text problem. It is a model with up to 11B parameters trained on a giant data set of 750 GB of clean English web text, or >1T tokens. To specify a task, a task specific prefix is added to the input sequence. While Bert adds another output layer on top of the transformer for each specific task, T5 applies the same model and decoding process to every task without changes in architecture. In some embodiments, The Stanford Question Answering Dataset (SQuAD) may be used in pre-training and fine-tuning T5 for a question answering task.

In other embodiments, BigBird, a transformer using a sparse attention mechanism linear in the number of tokens, which can handle much longer text sequences may be used. The ability of BigBird to model extended context benefits many NLP tasks. BigBird outperforms many other models on many NLP tasks such as question answering.

In some embodiments, the UMLS procedure concepts extracted from biomedical text may be mapped to CPT codes. UMLS procedure concepts often have an AUI in multiple ontologies included in UMLS (e.g., SNOMED), but less than 5% of UMLS procedure concepts are associated with the CPT taxonomy (i.e., have a CPT atom). Thus, more than 95% of UMLS procedure concepts cannot easily be mapped onto CPT codes. In some embodiments, the system maps UMLS procedure concepts to CPT codes using a shallow neural language model. The model generates CPT code and UMLS procedure concept embeddings. In some embodiments, a cosine similarity between CPT code embeddings and UMLS procedure concept embeddings aid in mapping UMLS procedure concepts to CPT codes.

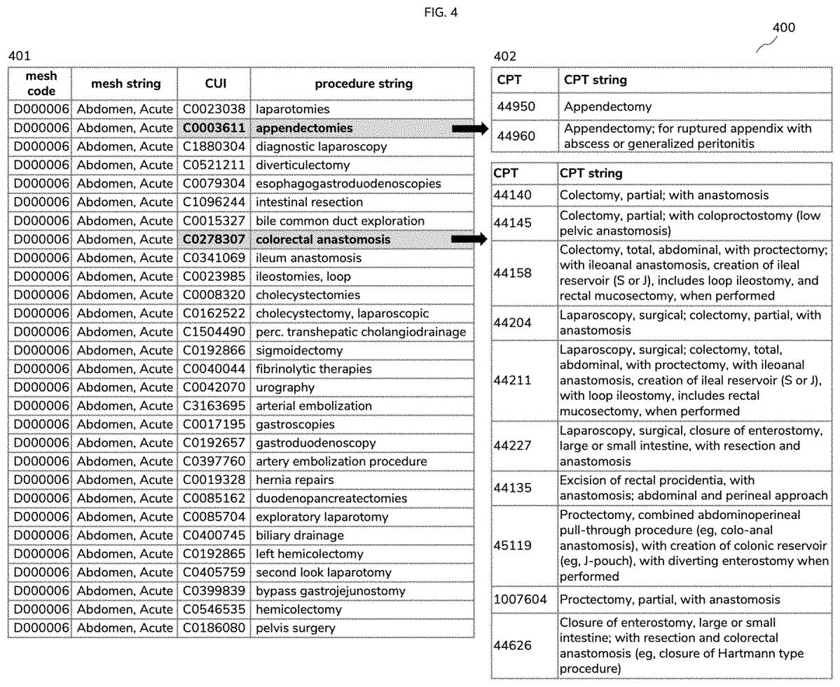

In further embodiments, the system employs additional protocols to ensure that the map of UMLS procedure concepts to CPT codes reaches complete accuracy. FIG. 4 depicts a mapping from a single condition (MeSH code D000006, Acute Abdomen) to multiple relevant procedure concepts (CUIs) in UMLS 401. Those concepts may then be mapped to multiple relevant CPT codes 402.

In further embodiments, deep learning language models allow the system to extract additional relevant information for each condition (e.g., affected organs, symptoms, comorbid conditions, and complications). This information may be used to enrich the MeSH ontology with new types of links. For example, the system can place a link between one condition and another condition that complicates the first condition. In some embodiments, the system may enrich the set of procedures of the first condition with the set of procedures of the complicating condition. In some embodiments, the system may use multiple separate mappings from MeSH disease terms to CPT codes, which are then combined and further refined using proprietary filters and other protocols. The result is each condition being associated with a final set of relevant CPT codes and a final set of relevant RxNorm codes.

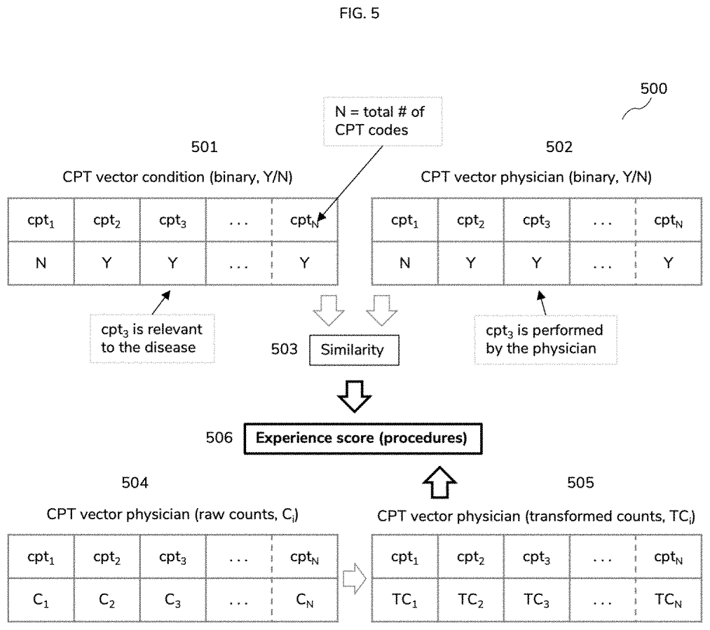

In some embodiments, each condition is associated with a set of CPT codes and a set of RxNorm codes. Each physician is likewise associated with a set of CPT codes and a set of RxNorm codes, obtained from claims data or other types of medical records data. Each set of CPT codes are represented as vectors in the vector space with the same dimensionality, wherein the number of dimensions equals the number of CPT codes. Each set of RxNorm codes are represented as vectors in the vector space with the same dimensionality, wherein the number of dimensions equals the number of RxNorm codes. Referring to FIG. 5, similarity 503 between a physician 502 and a condition 501 is defined as a cosine similarity between their respective code vectors (CPT or RxNorm). The physician procedural experience score 506 depends on (a) cosine similarity between the CPT vectors of the physician and the condition in question and (b) term frequency 505 (or term frequency-inverse document frequency) of the physician's relevant CPT codes 504. The physician drug regimen experience score depends on (a) cosine similarity between the RxNorm vectors of the physician and the condition in question and (b) term frequency (or term frequency-inverse document frequency) of the physician's relevant RxNorm codes.

In an alternate embodiment, the experience scores may combine CPT and RxNorm scores. In alternate embodiments, the experience score may be further refined by assigning different weights to different procedures based on the level of skill required and the importance of the procedure.

In some embodiments, the system may assign a higher weight to a procedure or drug regimen that requires a higher level of skill (e.g., surgical relative to pathological or radiological), is strongly recommended, is used as the first line of treatment, or is regarded as essential by expert consensus. In some embodiments, the system may use “ternary” instead of binary vectors to accomplish that goal. Ternary vector representation may allow physicians with fewer but more complex and/or essential procedures to obtain higher similarity weights than under a binary system.

In some embodiments, the term frequency-inverse document frequency statistic may be used to assign higher weights to rarer procedures or treatments because such procedures are considered more informative.

### Physician Name Disambiguation

The Innovation score depends on the ability to match physician names to author names on publications and investigator names on clinical trials. The biggest challenge in assigning publications to physicians is author name disambiguation. Since there are no unique author identifiers on published PubMed/PMC articles (it means there is no easy way of knowing which of the possibly many physicians with the name “John Smith” is the correct author), and since a large portion of publications doesn't even contain affiliation data for all authors, this task is exceedingly difficult. To solve this problem, in some embodiments, the system may create name spaces that include all PubMed identifiers (PMIDs) with the same first name, middle initial (if present) and last name. PMIDs in each name space may then be clustered into clusters that presumably represent single-author collections of articles using deep learning language models. Individual clusters may subsequently be assigned to the right physician using a protocol that considers author's location, organization, department, specialty, and PMID's Mesh terms.

In some embodiments, deep learning language models are trained for text classification for the specific task of deciding whether two PMIDs are authored by the same author. In some embodiments, a version of BioBERT specifically fine-tuned for this task may be used. Clustering PMIDs via BioBert allows the system to correctly assign PMIDs that lack substantial author affiliation data (organization, department, city, state).

### Authority Modeling

In some embodiments, as part of modeling a physician's expertise, the system models the authority of the physician. In further embodiments, the system scores authority based on the centrality of the physician within their referral network. PageRank (PR) used by Google measures centrality of web pages by analyzing a graph where web pages are nodes and hyperlinks are edges. A “query-dependent” PR considers links to topic-relevant pages only, and in case of a multi-term query, PR scores for individual terms are combined in a way that reflects the importance of each term. The Hyperlink-Induced Topic Search (HITS) model distinguishes between authorities, or pages that represent reliable resources and have numerous incoming links, and hubs, or pages with numerous outgoing links. The HITS model operates under the assumption that a great hub points to many good authorities, and a great authority is pointed to by many good hubs. The HITS algorithm is a natural fit for the physician referral network. The system may regard physicians receiving patient referrals as authorities, and physicians referring patients to other physicians as hubs. Most physicians act as both hubs and authorities.

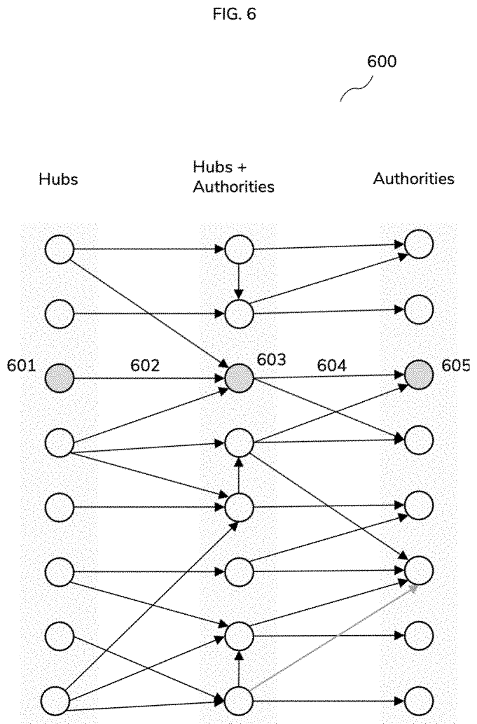

Referring to FIG. 6, a referral network 600 is depicted. Physician A 601 acts only as a hub, referring 602 to physician B 603. Physician B 603 is an authority and hub, as they receive referrals 602 from multiple physicians, including physician A 601, and refer 604 to other physicians, including physician C 605. Physician C 605 is only an authority, as they only receive referrals.

In some embodiments, the system uses the HITS hub/authority algorithm to model physician centrality in the US national referral networks. In further embodiments, the model has been extended by creating a “multi query-dependent” HITS algorithm that allows queries that involve more than one specialty and physicians that have more than one specialty. In addition, because a physician's ability as a hub is tied to his/her authority, it is reasonable to assume that good authorities make better referrals (i.e., by being experts, they are more likely to know and refer to other experts). In further embodiments, the model has been extended to reflect this assumption, resulting in a novel algorithm, “Intelligent Node Rank” (I-rank). I-rank uses an authority-enhanced hub score instead of the standard hub score, wherein a standard hub score is partially also a function of the authority score. In some embodiments, a standard hub score may also be amplified by some arbitrary “mastery” score (e.g., a score that reflects a physician's experience with a specific condition, or authority on the subject).

In some embodiments, I-rank may contain one or more other weights, for example weights that compensate for variations in the physician population across the US (e.g., across different hospital referral regions, or HRRs), cross-HRR referrals, type of referring entity etc.

### Quality Modeling

In some embodiments, as part of modeling a physician's experience, the system models the quality of the physician's care. In some embodiments, the system uses publicly available quality measures that may be normalized and averaged into a final quality score for each physician. In further embodiments, the system assigns the most weight to individual physician quality measures. In some embodiments, individual quality measures may comprise compliance with guidelines, mortality rates, complication rates, readmission rates etc. In further embodiments, the system also includes relevant hospital quality measures to account for the quality of hospitals with which the physician is affiliated. In some embodiments, the following quality related datasets may be used from the Centers for Medicare & Medicaid Services (CMS): Merit-Based Incentive Payment System (MIPS) final scores and performance category scores; the Overall Hospital Quality Star rating for hospitals which shows the quality of care a hospital may provide compared to other hospitals and summarizes as many as fifty measures in five measure groups (mortality, safety of care, readmission, patient experience, timely & effective care); and hospital value-based purchasing (VBP) program domain scores (clinical outcomes, community engagement, safety, efficiency, and cost reduction) and total performance scores. In some embodiments, individual components of the quality score may be separately weighted. For example, individual quality measures may be valued over hospital quality measures.

### Team Modeling

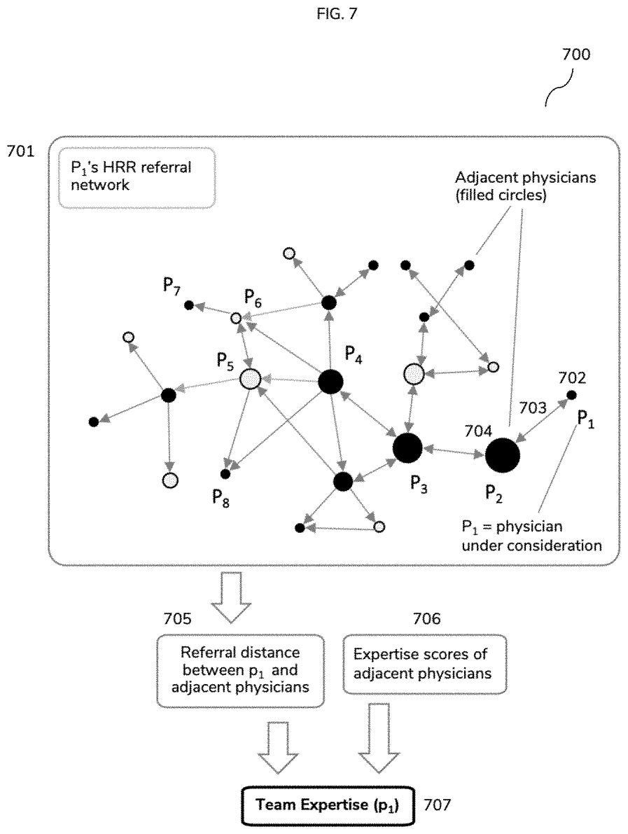

Physicians in the immediate network of the physician in question are relevant because most conditions require multiple specialties and a collaborative (patient-sharing) approach. Patients benefit from their physician's proximity to other experts; this was the motivation for the Team expertise score. More specifically, the Team expertise score considers the expertise of “adjacent physicians”. Adjacent physicians are defined as physicians who (a) practice in specialties relevant to the query; (b) belong in the same hospital referral region (HRR) as the physician in question; (c) either share patients with the physician in question or are positioned close to him/her in the same organization. FIG. 7 illustrates a method for determining a physician P1 ‘s expertise score 700 in accordance with an embodiment. In some embodiments, the physician P1’ s referral network within his/her HRR is mapped based on historical referrals among physicians in that HRR 701. Referral distance (a shortest referral path) 705 may be calculated between the physician in question and adjacent physicians. For example, physician P1 702 is directly adjacent to physician P2 704 as they have each referred to the other 703. In some embodiments, the shortest distance algorithm comprises calculating the referral distance in terms of the minimum number of referral links between two physicians. In some embodiments, physicians outside a certain threshold of referral distance may be removed from consideration. In some embodiments, referral directionality may determine the weight on edges in the referral graph. A final team rank 707 for the physician is calculated as a weighted sum of expertise scores of adjacent physicians 706, with weights being inversely proportional to the referral distance 705. In some embodiments, adjacent physicians who complement the skills of the physician in question (by being in a different relevant specialty or having complementary relevant procedural skills) are given more weight.

In some embodiments, one or more of the innovation, experience, authority, quality, and team scores may be combined to form a final score for the physician with respect to the designated medical condition. In some embodiments, the component scores forming the final score are separately weighted. In a further embodiment, the component scores are separately weighted based on the medical condition. For example, in response to a low-risk medical condition with a standard treatment, the system may weight experience over innovation. Conversely, in response to a high-risk medical condition for which a standard treatment may not work and access to novel treatments is beneficial, innovation may be weighted more than or as heavily as experience. In other embodiments, a user may be able to provide input relating to their preference in the weighting of the scores. In further embodiments, the final score may be used to determine a ranking of the physician in regard to other physicians in a region, hospital system, or based on any other searchable criteria.

In some embodiments, the resulting platform may be used by the referring physician or the patient (collectively, the “user”) looking to find the right doctor for their condition. In some embodiments, the user may be able to provide input comprising a medical condition or a symptom to start the search. In further embodiments, the user may be able to provide further search inputs comprising one or more of their location, maximum travel distance, an insurance provider, or a health system. In other embodiments, the user may be able to select preferences relating to specific traits in a physician, such as specialty, participation in clinical trials, or physician's innovation or experience scores. In further embodiments, the user may be able to rank physicians according to experience with a specific medical procedure. Various traits may be weighted by the user over a range of values. In some embodiments, the user may use of a slider, numerical entry, or other input method to adjust weights.

In some embodiments, the platform may be provided by an insurance provider. In further embodiments, the insurance provider may only provide results that are in network or flag results which are out of network.

In some embodiments, the platform may be provided by a hospital system. In further embodiments, the hospital system may only provide results that are within the hospital system or flag results which are outside of the hospital system.

In some embodiments, the platform may act as a care planning, or “concierge” service for a health company. For example, the platform displays a complete set of physician specialties relevant for a given condition and distinguishes primary (first to be visited) and secondary (potentially required) specialties. It ranks physicians in each relevant specialty. In some embodiments, the platform may provide an evolving plan for physician visitations that adapts to a particular individual's situation, including progress of the disease and prior treatments and their outcomes. In other embodiments, each stage of the plan may have a different set of weights in terms of how physicians in various specialties (e.g., medical versus surgical) are prioritized.

In some embodiments, on use of the platform, the user may be presented with a list of physicians. In some embodiments, the user may be able to re-sort the list based on factors such as specialty, overall ranking, clinical trial participation, individual scores (e.g., innovation or experience), location, health system, insurance acceptance, and/or new patient acceptance. In other embodiments, the user may be presented with a map of physicians in addition to or as an alternative to a list. In some embodiments, a photograph of a physician may be displayed. In other embodiments, a physician may be represented by a symbol that is determined based on the physician's expertise score, team expertise score, clinical trial participation and other factors.

In some embodiments, selecting a physician may present the user with additional information about the physician. As a non-limiting example, the user may be presented with information such as the physician's composite expertise score, individual scores, specialties, office address, office number, hospital affiliations, insurance accepted, website, types of visits accepted, and patient reviews. In some embodiments, the user may be able to access one or more relevant pieces of biomedical literature to which the physician contributed. In other embodiments, the user may be able to access and enroll in one or more relevant clinical trials led by the physician. In some embodiments, the user may be able to schedule or request an appointment or a remote consultation within the platform.

In some embodiments, the platform may act as a research tool. In some embodiments, clinical researchers and life sciences companies pursuing research and development may use the platform to find the top experts in specific clinical or disease areas. In further embodiments, the discovered experts may be mapped according to their location to discover where particular clinical research is being performed. Those skilled in the art will understand that the underlying data science model of physician expertise presented herein may prove useful in a variety of research endeavors. For example, researchers may be able to track innovative research in a particular specialty or clinical area.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object-oriented programming language such as Python, Java, Smalltalk, C++, C#, HTML, Angular, SQL, PostgreSQL, or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer, or entirely on the remote computer or server. In the latter scenario, the remote computer may be connected to the user's computer through any type of network, including LAN or WAN, or the connection may be made to an external computer (for example, through the Internet using an Internet Service Provider).

Aspects of the present invention are described herein with reference to flowchart illustrations and/or block diagrams of methods, apparatuses (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions or acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operations steps to be performed on the computer, other programmable apparatus, or other device to produce a computer implemented process, such that the instructions which are executed on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical functions. In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

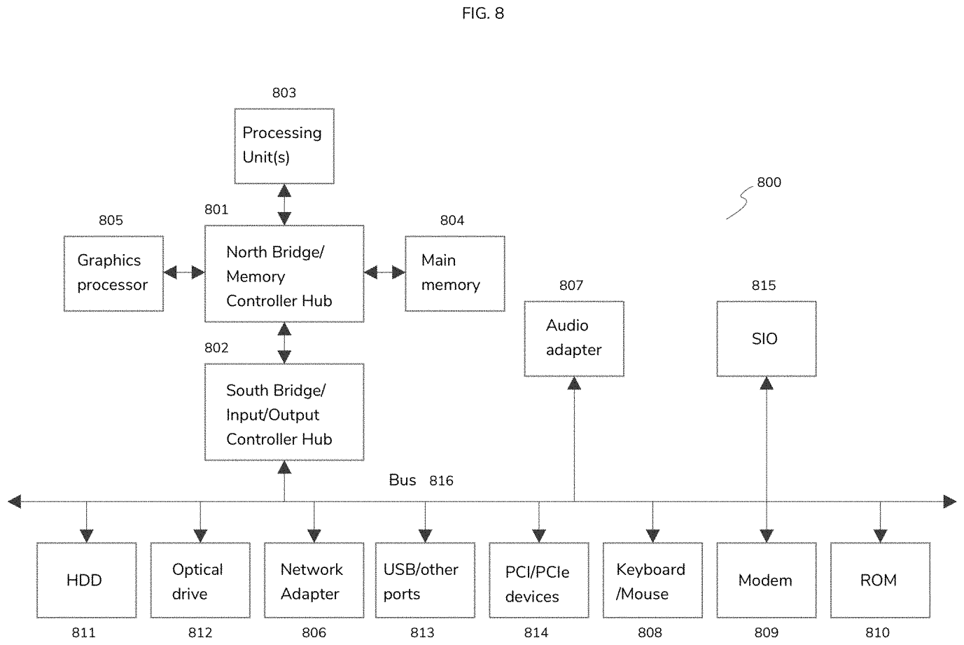

FIG. 8 is a block diagram of an example data processing system 800 in which aspects of the illustrative embodiments are implemented. Data processing system 800 is an example of a computer, such as a server or client, in which computer usable code or instructions implementing the process for illustrative embodiments of the present invention are located. In one embodiment, FIG. 8 may represent a server computing device, or cloud computing system, such as, for example, Amazon Web Services, Microsoft Azure, Google Cloud, Alibaba Cloud, Oracle Cloud, IBM Cloud, etc. In another embodiment, FIG. 8 may represent a client device, such as, for example, a personal computer, a table, smartphone, or any current or future electronic device capable of carrying out the embodiments disclosed herein.

In the depicted example, data processing system 800 can employ a hub architecture including a north bridge and memory controller hub (NB/MCH) 801 and south bridge and input/output (I/O) controller hub (SB/ICH) 802. Processing unit 803, main memory 804, and graphics processor 805 can be connected to the NB/MCH 801. Graphics processor 805 can be connected to the NB/MCH 801 through, for example, an accelerated graphics port (AGP) or PCI/PCIe port.

In the depicted example, a network adapter 806 connects to the SB/ICH 802. An audio adapter 807, keyboard and mouse adapter 808, modem 809, read only memory (ROM) 810, hard disk drive (HDD) 811, optical drive (for example, CD or DVD) 812, universal serial bus (USB) ports and other communication ports 813, and PCI/PCIe devices 814 may connect to the SB/ICH 802 through bus system 816. PCI/PCIe devices 814 may include Ethernet adapters, graphics processors 805, add-in cards, and PC cards for notebook computers. ROM 810 may be, for example, a flash basic input/output system (BIOS). The HDD 811 and optical drive 812 can use an integrated drive electronics (IDE) or serial advanced technology attachment (SATA) interface. A super I/O (SIO) device 815 can be connected to the SB/ICH 802.

An operating system can run on processing unit 803. The operating system can coordinate and provide control of various components within the data processing system 800. As a client, the operating system can be a commercially available operating system. An object-oriented programming system, such as the Java™ programming system, may run in conjunction with the operating system and provide calls to the operating system from the object-oriented programs or applications executing on the data processing system 800. As a server, the data processing system 800 can be an IBM® eServer™ System® running the Advanced Interactive Executive operating system, the Linux operating system, or any other server operating system known to one skilled in the art. The data processing system 800 can be a symmetric multiprocessor (SMP) system that can include multiple processors in the processing unit 803. Alternatively, a single processor system may be employed.

Instructions for the operating system, the object-oriented programming system, and applications or programs are located on storage devices, such as the HDD 811, and are loaded into the main memory 804 for execution by the processing unit 803. The processes for embodiments described herein can be performed by the processing unit 803 using computer usable program code, which can be located in a memory such as, for example, main memory 804, ROM 810, or in one or more peripheral devices.

A bus system 816 can be comprised of one or more busses. The bus system 816 can be implemented using any type of communication fabric or architecture that can provide for a transfer of data between different components or devices attached to the fabric or architecture. A communication unit such as the modem 809 or the network adapter 806 can include one or more devices that can be used to transmit and receive data.

Those of ordinary skill in the art will appreciate that the hardware depicted in FIG. 8 may vary depending on the implementation. Other internal hardware or peripheral devices, such as flash memory, equivalent non-volatile memory, or optical disk drives may be used in addition to or in place of the hardware depicted. Moreover, the data processing system 800 can take the form of any of several different data processing systems, including but not limited to, client computing devices, server computing devices, tablet computers, laptop computers, telephone or other communication devices, personal digital assistants, and the like. Essentially, data processing system 800 can be any known or later developed data processing system without architectural limitation.

The system and processes of the figures are not exclusive. Other systems, processes, and menus may be derived in accordance with the principles of embodiments described herein to accomplish the same objectives. It is to be understood that the embodiments and variations shown and described herein are for illustration purposes only. Those skilled in the art may implement modifications to the current design, without departing from the scope of the embodiments. As described herein, the various systems, subsystems, agents, managers, and processes can be implemented using hardware components, software components, and/or combinations thereof. No claim element herein is to be construed under the provisions of 35 U.S.C. 112(f) unless the element is expressly recited using the phrase “means for.”

Although the invention has been described with reference to exemplary embodiments, it is not limited thereto. Those skilled in the art will appreciate that numerous changes and modifications may be made to the preferred embodiments of the invention and that such changes and modifications may be made without departing from the true spirit of the invention. It is therefore intended that the appended claims be construed to cover all such equivalent variations as they fall within the true spirit and scope of the invention.

What is claimed is:

1. A system for producing a clinically relevant measure of physician expertise comprising:
a processor; and
a non-transitory, computer-readable storage medium in operable communication with the processor, wherein the computer-readable storage medium contains one or more programming instructions that, when executed, cause the processor to:
receive physician's data relating to one or more physicians,
receive one or more datasets of biomedical corpora, clinical guidelines, clinical trials databases, biomedical ontologies, and other clinical resources,
receive input comprising at least one of one or more medical conditions and one or more symptoms,
determine one or more subsets of the one or more datasets relating to at least one of the one or more physicians,
generate one or more semantically similar terms to the input,
generate innovation information comprising a statistical analysis of the input and one or more semantically similar terms in the one or more subsets,
generate experience information comprising a statistical analysis of the context relating to the input within the one or more subsets,
generate innovation scores, for each of the one or more physicians, based on the innovation information,
generate experience scores, for each of the one or more physicians, based on the experience information, and
score the one or more physicians' expertise as it relates to the medical condition based on a combination of the innovation scores and the experience scores.

2. The system of claim 1, wherein the one or more programming instructions that, when executed, cause the processor to score the one or more physicians' expertise with respect to the medical condition based on a combination of the innovation scores and the experience scores include one or more programming instructions that, when executed, cause the processor to score the one or more physicians' expertise further based on an authority score.

3. The system of claim 1, wherein the one or more programming instructions that, when executed, cause the processor to score the one or more physicians' expertise with respect to the medical condition based on a combination of the innovation scores and the expertise scores include one or more programming instructions that, when executed, cause the processor to score the one or more physicians' expertise further based on a quality score.

4. The system of claim 1, wherein the one or more programming instructions that, when executed, cause the processor to score the one or more physicians' expertise with respect to the medical condition based on a combination of the innovation scores and the expertise scores include one or more programming instructions that, when executed, cause the processor to score the one or more physicians' expertise further based on a team score.

5. The system of claim 1, wherein the one or more programming instructions that, when executed, cause the processor to determine one or more subsets of the one or more datasets relating to at least one of the one or more physicians further comprise one or more programming instructions that, when executed, cause the processor to perform name disambiguation on the one or more physicians.

6. The system of claim 1, wherein the one or more programming instructions that, when executed, cause the processor to generate an innovation score comprise one or more programming instructions that, when executed, cause the processor to calculate a term frequency for at least one of the inputs and the one or more semantically similar terms.

7. The system of claim 1, wherein the one or more programming instructions that, when executed, cause the processor to generate one or more semantically similar terms to the input comprise one or more programming instructions that, when executed, cause the processor to exploit the taxonomical structure of the Medical Subject Headings ontology.

8. The system of claim 1, wherein the statistical analysis of the context relating to the input comprises at least one of the following algorithms: shallow neural models, deep learning models, natural language processing, word2vec, GloVE, biowordvec, cui2vec, transformer-based models, BERT, BioBERT, T5, and BigBird.

9. The system of claim 1, wherein the statistical analysis of the context relating to the input comprises determining guidelines, procedures, and drug regimens for diagnosing and treating the input.

10. The system of claim 1, wherein the statistical analysis of the context relating to the input comprises mapping UMLS procedure concepts onto CPT codes.

11. A method for producing a clinically relevant measure of physician expertise comprising:
receiving physician's data relating to one or more physicians,
receiving one or more datasets of biomedical corpora, clinical guidelines, clinical trials databases, biomedical ontologies, and other clinical resources,
receiving input comprising at least one of one or more medical conditions and one or more symptoms,
determining one or more subsets of the one or more datasets relating to at least one of the one or more physicians,
generating one or more semantically similar terms to the input,
generating innovation information comprising a statistical analysis of the input and the one or more semantically similar terms in the one or more subsets,
generating experience information comprising a statistical analysis of the context relating to the input within the one or more subsets,
generating innovation scores, for each of the one or more physicians, based on the innovation information,
generating experience scores, for each of the one or more physicians, based on the experience information, and
scoring the one or more physicians' expertise as it relates to the medical condition based on a combination of the innovation scores and the expertise scores.

12. The method of claim 11, wherein scoring the one or more physicians' expertise with respect to the medical condition based on a combination of the innovation scores and the experience scores further comprises scoring the one or more physicians' expertise further based on an authority score.

13. The method of claim 11, wherein scoring the one or more physicians' expertise with respect to the medical condition based on a combination of the innovation scores and the expertise scores further comprises scoring the one or more physicians' expertise further based on a quality score.

14. The method of claim 11, wherein scoring the one or more physicians' expertise with respect to the medical condition based on a combination of the innovation scores and the experience scores further comprises scoring the one or more physicians' expertise further based on a team score.

15. The method of claim 11, wherein determining one or more subsets of the one or more datasets relating to at least one of the one or more physicians further comprises performing name disambiguation on the one or more physicians.

16. The method of claim 11, wherein generating an innovation score comprises calculating a term frequency for at least one of the inputs and the one or more semantically similar terms.

17. The method of claim 11, wherein generating one or more semantically similar terms to the input comprises exploiting the taxonomical structure of the Medical Subject Headings ontology.

18. The method of claim 11, wherein the statistical analysis of the context relating to the input comprises at least one of the following algorithms: shallow neural models, deep learning models, natural language processing, word2vec, GloVE, biowordvec, cui2vec, transformer-based models, BERT, BioBERT, T5, and BigBird.

19. The method of claim 11, wherein the statistical analysis of the context relating to the input comprises determining guidelines, procedures, and drug regimens for diagnosing and treating the input.

20. The method of claim 11, wherein the statistical analysis of the context relating to the input comprises mapping UMLS procedure concepts onto CPT codes.
